(19) United States
(12) Patent Application Publication
(10) Pub. No.: US 2024/0268990 A1
(43) Pub. Date: Aug. 15, 2024

(54) FLUID COLLECTION ASSEMBLIES EXHIBITING A RELATIVELY THIN SHAPE

(71) Applicant: PUREWICK CORPORATION, Covington, GA (US)

(72) Inventor: Camille Rose Newton, Bonsall, CA (US)

(21) Appl. No.: 18/569,711

(22) PCT Filed: Jun. 23, 2022
(86) PCT No.: PCT/US2022/034744

§ 371 Date: Dec. 13, 2023

Related U.S. Application Data
(60) Provisional application No. 63/215,017, filed on Jun. 25, 2021.

(51) Int. Cl. A61F 5/451

(52) U.S. Cl. CPC A61F 5/451

(57) ABSTRACT

An example fluid collection assembly includes a fluid impermeable barrier defining at least a chamber, at least one opening. and a fluid outlet. The fluid collection assembly also includes a fluid permeable support and a fluid permeable membrane disposed in the chamber. The fluid collection assembly exhibits a relatively thin shape which allows the fluid collection assembly to be used more effectively in more situations than at least some conventional fluid collection assemblies.

### CROSS-REFERENCE TO RELATED APPLICATIONS

This application claims priority to U.S. Provisional Patent application No. 63/215,017 filed on Jun. 25, 2021, entitled FLUID COLLECTION ASSEMBLIES EXHIBITING A RELATIVELY THIN SHAPE, the disclosure of which is incorporated by reference herein, in its entirety.

### BACKGROUND

A patient may have limited or impaired mobility such that typical urination processes are challenging or impossible. For example, the patient may have surgery or a disability that impairs mobility. In another example, the patient may have restricted travel conditions such as those experience by pilots, drivers, and workers in hazardous areas. Additionally, fluid collection from the patient may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

### SUMMARY

In an embodiment, a fluid collection assembly is disclosed. The fluid collection assembly includes a fluid impermeable barrier defining at least a chamber, at least one opening, a fluid outlet, and a sump. The fluid collection assembly also includes a fluid permeable support, a fluid permeable membrane disposed on at least a portion of the fluid permeable support and extending across the opening, and a conduit connected to the fluid outlet, the conduit including an inlet disposed in or adjacent to the sump. The fluid collection assembly exhibits a relatively thin shape.

In an embodiment, a fluid collection system is disclosed. The fluid collection system includes a fluid collection assembly. The fluid collection assembly includes a fluid impermeable barrier defining at least a chamber, at least one opening, a fluid outlet, and a sump. The fluid collection assembly also includes a fluid permeable support, a fluid permeable membrane disposed on at least a portion of the fluid permeable support and extending across the opening, and a conduit connected to the fluid outlet, the conduit including an inlet disposed in or adjacent to the sump. The fluid collection assembly exhibits a relatively thin shape. The fluid collection system also includes a fluid storage container and a vacuum source. The chamber of the fluid collection assembly is in fluid communication with the fluid storage container and the vacuum source via one or more conduits.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C, respectively, according to an embodiment.

FIG. 2 is a cross-sectional schematic of a fluid collection assembly, according to an embodiment.

FIG. 3 is a cross-sectional schematic of a fluid collection assembly exhibiting a generally non-rectangular cross-sectional shape, according to an embodiment.

FIGS. 4-6 are cross-sectional schematics of different fluid collection assemblies including a conduit that at least partially extends outside of the chamber, according to different embodiments.

FIGS. 7-11 are top plan view of fluid collection assemblies exhibiting at least one in-plane bend, according to different embodiments.

FIG. 12A is an isometric view of a fluid collection assembly that includes a fluid impermeable barrier that does not exhibit a shape that generally corresponds to the shape of the fluid permeable membrane, according to an embodiment.

FIG. 12B is a cross-sectional schematic of the fluid collection assembly taken along plane 12B-12B.

FIG. 13A is an isometric view of a fluid collection assembly, according to an embodiment.

FIG. 13B is a cross-sectional schematic of the fluid collection assembly of FIG. 13A.

FIG. 13C is an isometric view of the fluid collection assembly of FIG. 13A, shown being worn by a user, according to an embodiment.

FIG. 14 is an isometric view of a fluid collection assembly being worn by a user, according to an embodiment.

FIG. 15A is an isometric view of a fluid collection assembly being worn by a user, according to an embodiment.

FIG. 15B is a cross-sectional schematic of the fluid collection assembly of FIG. 15A

FIG. 16 is an isometric view of a fluid collection assembly being worn by a user, according to an embodiment.

FIG. 17 is a block diagram of a fluid collection system for fluid collection, according to an embodiment.

### DETAILED DESCRIPTION

Embodiments are directed to fluid collection assemblies, fluid collection systems including the same, and methods of forming and using the same. An example fluid collection assembly includes a fluid impermeable barrier defining at least a chamber, at least one opening, and a fluid outlet. The fluid collection assembly also includes a fluid permeable support and a fluid permeable membrane disposed in the chamber. The fluid collection assembly exhibits a relatively thin shape which, as will be discussed in more detail below, allows the fluid collection assembly to be used effectively in more situations than at least some other conventional fluid collection assemblies.

During use, the fluid collection assembly (e.g., the opening) may be disposed adjacent to a fluid source, such as adjacent to a urethral opening, a wound, or other fluid source. Fluids discharged from the fluid source may flow through the opening and into the chamber. For example, the fluids may be received into the fluid permeable membrane and flow into the fluid permeable support. The fluids may generally flow through the fluid permeable support towards the sump and the inlet of the conduit due to a suction provided to the chamber from the conduit or gravity. The fluids may be removed from the chamber by entering the inlet of the conduit, flowing through the conduit, and being deposited in the fluid storage container. As such, the fluid collection assembly may maintain the region about the fluid source relatively dry.

As previously discussed, the fluid collection assemblies disclosed herein may be used more effectively in more situations than other conventional fluid collection assemblies due to the shape of the fluid permeable support. For example, at least some fluid collection assemblies may exhibit a particular shape that enables the conventional fluid collection assembly to receive fluids from a specific fluid source. For instance, a conventional fluid collection assembly configured to receive fluids from a female urethral opening may exhibit a generally cylindrical shape while a conventional fluid collection assembly configured to receive fluids from a male urethral opening may exhibit a shape that is configured to receive a penis (e.g., a condom-like or bag-like shape). Such conventional fluid collection assemblies may be effective at removing fluids from their specific fluid source. However, such conventional fluid collection assemblies may be ineffective (e.g., may not receive substantially all of the fluids or may allow excessive fluids received thereby to easily leak therefrom) when used with other fluid sources (e.g., blood, blood serum, other serosanguineous drainage, vaginal discharge fluids, semen, stool, sweat, saliva, water, sewage, or any other fluid). Such conventional fluid collection assemblies may be ineffective when collecting fluids from the other fluid source for a variety of reasons. In an example, the conventional fluid collection assemblies do not exhibit a shape that corresponds to the region surrounding the other fluid sources which causes gaps to form between the conventional fluid collection assembly and the region surrounding the other fluid sources. Such gaps allow fluids to flow therein without being received into the conventional fluid collection assembly. In an example, the porous materials of the conventional fluid collection assemblies may be spaced from the fluid source which may result in pooling of the fluids (which may cause patient discomfort or unsanitary conditions), prevent the fluids from being received into the conventional fluid collection assembly, or allow the fluids to easily leak from the conventional fluid collection assembly. In an example, the conventional fluid collection assembly may have difficulty maintaining a positioned adjacent to the fluid source.

The fluid collection assemblies disclosed herein exhibit a relatively thin shape. As used herein, the relatively thin shape refers to a shape that exhibits a length measured parallel to a longitudinal axis of the fluid collection assembly and a width measured perpendicular to the length that is significantly greater (e.g., about 1.5 times or greater, about 2 times or greater, about 3 times or greater, about 4 times or greater, about 5 times or greater, about 7.5 times or greater, about 10 times or greater) than a thickness of the fluid collection assembly, wherein the thickness is measured perpendicular to the length and width. In other words, the thickness of the fluid collection assembly is significantly less than the length and the width of the fluid collection assembly. In some embodiments, the thickness of the fluid collection assembly is bendable and is thin relative to the thickness of a finger of a human user of the assembly. When the fluid collection assemblies disclosed herein exhibit a relatively thin shape, at least one of the fluid impermeable barrier exhibits a hollow relatively thin shape, the fluid permeable support exhibits a relatively thin shape, or the fluid permeable membrane exhibits a relatively thin shape. In an embodiment, the relatively thin shape of the fluid collection assemblies disclosed herein may be configured such that the length and width thereof lie in a plane. In an embodiment, the relatively thin shape of the fluid collection assemblies disclosed herein may include a generally elongated right rectangular prism; an elongated shape exhibiting a generally rectangular, a generally trapezoidal, a generally oblong, a generally oval, or a generally ellipsoidal cross-sectional shape; a generally V-like, a generally Y-like, a generally zig-zag, or a generally annular shape; or any other suitable relatively thin shape.

In an embodiment, the relatively thin shape of the fluid collection assemblies disclosed herein allows the fluid collection assemblies to be easily bent or shaped to exhibit a shape due to the relative thinness thereof. Easily bending and shaping the fluid collection assemblies allows the fluid collection assemblies to exhibit a shape that generally corresponds to the shape of the region about the fluid source. For example, the fluid collection assemblies may be bent or shaped to fit adjacent to the vulva or bend around a tip of a penis. As such, the fluid collecting assemblies disclosed herein may be easily manipulated to a shape that corresponds to the shape of the region about the fluid source which prevents the formation of gaps, decreases the distance from the porous materials (e.g., fluid permeable support and fluid permeable membrane) to the fluid source, and helps maintain the position of the fluid collection assembly adjacent to the fluid source.

The fluid collection assemblies disclosed herein may be used effectively with a variety of different fluid sources. Examples of fluid sources that the fluid collection assemblies may be used with include urethral openings (e.g., female urethral opening or male urethral opening), vagina, a wound, skin (e.g., to remove sweat from fingers, such as from a surgeons fingers during surgery), a leaking plumbing, or any other fluid source.

As such, the fluids that the fluid collection assemblies disclosed herein may receive include urine, blood, blood serum, other serosanguineous drainage, vaginal discharge fluids, semen, stool, sweat, saliva, water, sewage, or any other fluid.

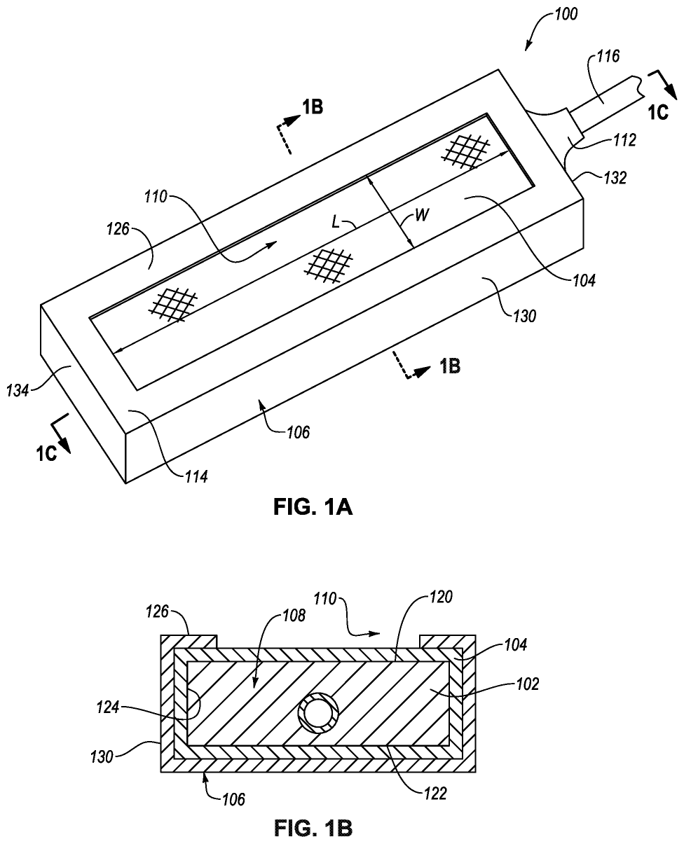

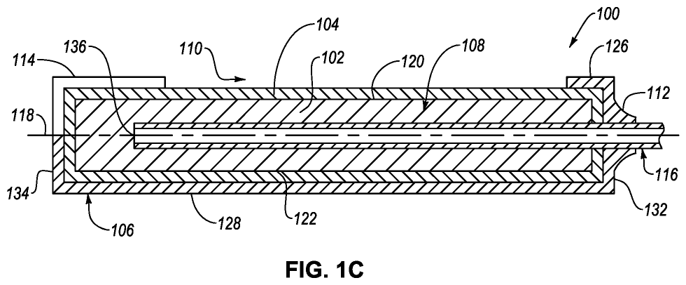

FIG. 1A is an isometric view of a fluid collection assembly 100, according to an embodiment. FIGS. 1B and 1C are cross-sectional schematics of the fluid collection assembly 100 taken along planes 1B-1B and IC-1C, respectively, according to an embodiment. The fluid collection assembly 100 includes a fluid impermeable barrier 106, a fluid permeable support 102, and a fluid permeable membrane 104. The fluid permeable membrane 104 may or may not wrap around the fluid permeable support 102. The fluid impermeable barrier 106 may define at least a chamber 108, an opening 110, a fluid outlet, and a sump 114. The fluid permeable support 102 and the fluid permeable membrane 104 are disposed in the chamber 108 such that the fluid permeable membrane 104 extends across the opening 110. The fluid collection assembly 100 also includes a conduit 116 that is in fluid communication with the chamber 108 such that one or more fluids received into the chamber 108 may be removed therefrom using the conduit 116.

The fluid permeable support 102 includes a first surface 120, a second surface 122, and one or more edges 124 extending from the first surface 120 to the second surface 122. The first surface 120 generally extends across the opening 110 formed in the fluid impermeable barrier 106. The fluid permeable support 102 may exhibit a thickness t measured from the first surface 120 to the second surface 122. The thickness t may be selected to be about 3 cm or less, about 2.5 cm or less, about 2 cm or less, about 1.75 cm or less, about 1.5 cm or less, about 1.25 cm or less, about 1 cm or less, about 9 mm or less, about 8 mm or less, about 7 mm or less, about 6 mm or less, about 5 mm or less, about 4 mm or less, about 3 mm or less, or in ranges of about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 1.25 cm, about 9 m to about 1.5 cm, about 1 cm to about 1.75 cm, about 1.25 cm to about 2 cm, about 1.5 cm to about 2.5 cm, or about 2 cm to about 3 cm. Generally, decreasing the thickness t may make bending and shaping the fluid collection assembly 100 easier. However, decreasing the thickness t of the fluid permeable support 102 may decrease the volume of fluids that may be present (e.g., flow, collected, or temporarily stored within) at any given moment within the support 102. As such, the thickness t may be selected based on balancing the need to bend and shape the fluid collection assembly 100 and the desired volume of fluids that may be present in the fluid permeable support 102 at any given time. It is noted that increasing the other dimensions (length and width) of the fluid permeable support 102 or increasing the rate at which the fluids are removed from the fluid permeable support 102 may allow the thickness t to be decreased even more.

In an embodiment, at least one of the first surface 120, the second surface 122, or the one or more edges 124 may exhibit a generally planar shape, such as a generally elongated rectangular shape. The generally rectangular shape may allow the first surface 120, the second surface 122, and/or the edges 124 to be positioned adjacent to a planar or generally planar fluid source while also allowing surface surfaces to be bent or otherwise shaped to conform to a non-generally planar fluid source.

The fluid permeable support 102 exhibits a relatively thin shape, such as any of the relatively thin shapes disclosed herein. In an embodiment, as illustrated, the fluid permeable support 102 may exhibit a generally rectangular prism shape. When the fluid permeable support 102 exhibits the generally rectangular prism shape, the fluid permeable support 102 may exhibit a generally rectangular cross-sectional shape (e.g., generally square cross-sectional shape) when the cross-section is taken parallel (as shown in FIG. 1B) and perpendicular (as shown in FIG. 1C) to a longitudinal axis 118 of the fluid collection assembly 100. For example, the first surface 120, the second surface 122, and the edges 124 may exhibit a generally rectangular shape when the fluid permeable support 102 exhibit a generally rectangular prism shape. The fluid collection assembly 100 may exhibit a shape that generally corresponds to the shape of the fluid permeable support 102. As such, the fluid collection assembly 100 may exhibit a generally rectangular prism shape that generally corresponds to the generally rectangular prism shape of the fluid permeable support 102. The generally rectangular prism shape of the fluid collection assembly 100 and the fluid permeable support 102 allows the fluid collection assembly 100 to be used effectively in a variety of situations, as previously discussed, For example, the generally rectangular prism shape of the fluid collection assembly 100 allows the fluid collection assembly 100 to be easily bent or shaped and allow the fluid permeable membrane 104 to extend across and be adjacent to the opening 110 regardless of how the fluid collection assembly 100 is shaped and bent.

The fluid permeable support 102 is configured to support the fluid permeable membrane 104 since the fluid permeable membrane 104 may be formed from a relatively foldable, flimsy, or otherwise easily deformable material. For example, the fluid permeable support 102 may be positioned such that the fluid permeable membrane 104 is disposed between the fluid permeable support 102 and the fluid impermeable barrier 106. As such, the fluid permeable support 102 may support and maintain the position of the fluid permeable membrane 104. The fluid permeable support 102 may include any material that may wick the fluids, such as any of the fluid permeable membrane materials disclosed herein above. For example, the fluid permeable membrane material(s) may be utilized in a more dense or rigid form than in the fluid permeable membrane 104 when used as the fluid permeable support 102. The fluid permeable support 102 may be formed from any fluid permeable material that is less deformable than the fluid permeable membrane 104. For example, the fluid permeable support 102 may include a porous polymer (e.g., nylon, polyester, polyurethane, polyethylene, polypropylene, etc.) structure or an open cell foam (e.g., spun nylon fiber), or nonwoven material. In some examples, the fluid permeable support 102 may be formed from a natural material, such as cotton, wool, silk, or combinations thereof. In such examples, the material may have a coating to prevent or limit absorption of fluid into the material, such as a water repellent coating. In some examples, the fluid permeable support 102 may be formed from fabric, felt, gauze, or combinations thereof.

In an embodiment, the fluid permeable support 102 may be formed from a sheet of porous material, such as a sheet of spun nylon fibers or a nonwoven sheet. The relatively thin shape of the fluid permeable membrane 102 (e.g., the generally rectangular prism shape of the fluid permeable support 102) allows the fluid permeable support 102 to be formed by merely cutting the sheet of porous material without needing to perform any other shaping procedure. As such, forming the fluid permeable support 102 to exhibit the generally rectangular prism shape may facilitate formation of the fluid permeable support 102.

The fluid permeable membrane 104 may be composed to wick the fluids away from the opening 106, thereby pulling the fluids into the chamber and preventing the fluids and the vacuum from escaping the chamber 108. In an embodiment, the fluid permeable membrane 104 may include any material that may wick the fluids. For example, the fluid permeable membrane 104 may include fabric, such as a gauze (e.g., a silk, linen, or cotton gauze), another soft fabric, paper, or another smooth fabric. Forming the fluid permeable membrane 104 from gauze, soft fabric, and/or smooth fabric may reduce chaffing caused by the fluid collection assembly 100. In some examples, the fluid permeable membrane 104 may be optional.

The fluid permeable support 102 and/or the fluid permeable membrane 104 may also have an ability to wick and/or enable flow of the fluids therethrough such as to move the fluids inwardly from the outer surface of the fluid collection assembly 100.

In an embodiment, at least a portion of the fluid permeable support 102 and/or the fluid permeable membrane 104 may be a porous material configured to wick and/or allow flow of the fluids away from the opening 110, thereby preventing fluids and vacuum from escaping the chamber 108. The porous material (e.g., one or more of the fluid permeable support 102 or the fluid permeable membrane 104) may not include absorption of the fluids into the wicking material. Put another way, substantially no absorption of the fluids into the one or more of the fluid permeable support 102 or the fluid permeable membrane 104 may take place after the wicking material is exposed to the fluids. While no absorption is desired, the term “substantially no absorption” may allow for nominal amounts of absorption of the fluids into the porous material (e.g., absorbency), such as about 30 wt % of the dry weight of the wicking material, about 20 wt % or less, about 15 wt % or less, about 10 wt % or less, about 7 wt % or less, about 5 wt % or less, about 3 wt % or less, about 2 wt % or less, about 1 wt % or less, or about 0.5 wt % or less of the dry weight of the wicking material. In some embodiments, at least one of the fluid permeable support 102 or the fluid permeable membrane 104 includes non-wicking materials (e.g., absorbent and/or adsorbent materials).

The fluid permeable membrane 104 is disposed on at least a portion of the first surface 120 of the fluid permeable support 102. In some embodiments, the fluid permeable membrane 104 may also be disposed on all or substantially all of the first surface 120, at least a portion (e.g., all) of the second surface 122 of the fluid permeable support 102, and/or at least a portion (e.g., all) of the edges 124 of the fluid permeable support 102.

In an embodiment, the fluid permeable membrane 104 may extend completely around a circumference of the fluid permeable support 102, as shown in FIG. 1B). In such an embodiment, the fluid permeable membrane 104 may exhibit a generally tubular shape. Extending the fluid permeable membrane 104 completely around the circumference of the fluid permeable support 102 may allow the fluid permeable membrane 104 to be secured to the fluid permeable support 102 and maintain the fluid permeable membrane 104 extending across the opening 110 without using adhesives, sewing, or other attachment methods. However, as will be discussed in more detail below, the fluid permeable membrane 104 may only extend around a portion of the fluid permeable support 102.

As previously discussed, the fluid collection assembly 100 includes a fluid impermeable barrier 106. The fluid impermeable barrier 106 may be formed of any suitable fluid imporous material(s), such as a fluid impermeable polymer (e.g., silicone, polypropylene, polyethylene, polyethylene terephthalate, a polycarbonate, etc.), a metal film, natural rubber, another suitable material, or combinations thereof. The fluid impermeable barrier 106 substantially prevents the fluids from passing through the fluid impermeable barrier 106. In an example, the fluid impermeable barrier 106 may be air permeable and fluid impermeable. In such an example, the fluid impermeable barrier 106 may be formed of a hydrophobic material that defines a plurality of pores. At least a surface of the fluid impermeable barrier 106 that may contact the individual may be formed from a soft and/or smooth material (e.g., silicone), thereby reducing chaffing. In an embodiment, the fluid impermeable barrier 106 may be formed from a flexible material, such as silicone, which allows the fluid impermeable barrier 106 to be bent into a shape that conforms the anatomy of the individual.

The fluid impermeable barrier 106 exhibits a shape that generally corresponds to the shape of the fluid permeable support 102. As such, in the illustrated embodiment, the fluid impermeable barrier 106 may exhibit a generally rectangular prism shape that corresponds to the generally rectangular prism shape of the fluid permeable support 102. For example, the fluid impermeable barrier 106 may exhibit a front surface 128 defining the opening 110, a back surface 128 opposite the front surface 126, and at least one lateral surface 130 extending from the front surface 126 to the back surface 128. The front surface 126 is adjacent to the first surface 120 and, as such, the front surface 126 may exhibit a shape (e.g., generally rectangular shape) that generally corresponds to the shape of the first surface 120. The back surface 128 is adjacent to the back surface 122 and, as such, the back surface 128 may exhibit a shape (e.g., generally rectangular shape) that generally corresponds to the shape of the first surface 120. The latera surface 130 is adjacent to the edges 126 of the fluid permeable support 102 and, as such, the lateral surface 130 may exhibit a shape (e.g., generally rectangular shape) that corresponds to the shape of the edges 126.

The opening 110 provides an ingress route for fluids to enter the chamber 108. The opening 110 may be defined by the fluid impermeable barrier 106 such as by an inner edge of the fluid impermeable barrier 106. For example, the opening 110 is formed in and extends through the fluid impermeable barrier 106 thereby enabling fluid(s) to enter the chamber 108 from outside of the fluid collection assembly 100. The opening 110 may be an elongated hole in the fluid impermeable barrier 106. For example, the opening 110 may be defined as a cut-out in the fluid impermeable barrier 106. The opening 110 may be located and shaped to be positioned adjacent to a fluid source, such as a female urethral opening, a wound, an ostomy site, etc.

The fluid collection assembly 100 may be positioned proximate to the fluid source and fluid may enter the chamber of the fluid collection assembly 100 via the opening 110. The fluid collection assembly 100 is configured to receive the fluids into the chamber 108 via the opening 110.

The opening 110 is formed in the front surface 128 of the fluid impermeable barrier 106. The opening 110 may exhibit a shape that generally corresponds to the shape of the front surface 128 to maximize the amount of fluids that may flow into the chamber 108 through the opening 110. In other words, the opening 110 may exhibit a shape that generally corresponds to the shape of the first surface 120.

The opening 110 may exhibit a length L and a width W. The length L and the width W may be measured parallel and perpendicular, respectively, to a path of the opening 110. For example, the length L and the width W may be measured parallel and perpendicularly, respectively, to the longitudinal axis 118 when the opening 110 extends generally parallel to the longitudinal axis 118. The length L may be selected to be about 1 cm to about 3 cm, about 2 cm to about 4 cm, about 3 cm to about 5 cm, about 5 cm to about 7 cm, about 6 cm to about 8 cm, about 7 cm to about 9 cm, about 8 cm to about 10 cm, about 9 cm to about 12 cm, about 10 cm to about 13.5 cm, about 12 cm to about 15 cm, about 13.5 cm to about 17.5 cm, about 15 cm to about 20 cm, about 17.5 cm to about 22.5 cm, about 20 cm to about 25 cm, or greater than about 22.5 cm. The width W may be selected to be about 0.5 cm to about 1 cm, bout 0.75 cm to about 1.25 cm, about 1 cm to about 1.5 cm, about 1.25 cm to about 1.75 cm, about 1.5 cm to about 2 cm, about 1.75 cm to about 2.5 cm, about 2 cm to about 3 cm, about 2.5 cm to about 3.5 cm, about 3 cm to about 4 cm, about 3.5 cm to about 4.5 cm, about 4 cm to about 5 cm, about 4.5 cm to about 6 cm, or greater than about 5 cm. Generally, increasing the length L and the width W of the opening 110 allows a greater quantity of the fluids to flow into the chamber 108, be received by the fluid permeable membrane 104, and flow into the fluid permeable support 102. However, increasing the length L and width W dissipates a suction applied from the conduit 116 to the chamber 108 which decreases the effectiveness of the suction at removing fluids from the chamber 108. As such, the length L may be limited to about 3 cm to about 20 cm and/or the width W may be limited to about 1 cm to about 5 cm.

The opening 110 may exhibit a surface area of about 3 cm² to about 5 cm², about 4 cm² to about 6 cm², about 5 cm² to about 10 cm², about 8 cm² to about 15 cm², about 10 cm² to about 20 cm², about 15 cm² to about 25 cm², about 20 cm² to about 25 cm², about 20 cm² to about 30 cm², about 25 cm² to about 35 cm², about 30 cm² to about 45 cm², about 40 cm² to about 55 cm², about 50 cm² to about 65 cm², about 60 cm² to about 75 cm², about 70 cm² to about 85 cm², about 80 cm² to about 95 cm², about 90 cm² to about 105 cm², or greater than 100 cm². The surface area of the opening 110 depends on the length L and the width W of the opening 110. As such, increasing the surface area of the opening 110 allows a greater quantity of the fluids to flow into the chamber 108, be received by the fluid permeable membrane 104, and flow into the fluid permeable support 102. However, increasing the surface area dissipates a suction and increase air flow applied from the conduit 116 to the chamber 108 which decreases the effectiveness of the suction at removing fluids from the chamber 108.

As previously discussed, the fluid impermeable barrier 106 may define fluid outlet configured to remove fluids from the chamber 108. The fluid outlet is distinct from the opening 110 and the valve 120. In some examples, the fluid outlet is sized to receive the conduit 116. The conduit 116 may be disposed in the chamber 108 via the fluid outlet. The fluid outlet may be sized and shaped to form an at least substantially fluid tight seal against the conduit 116 or the at least one conduit substantially preventing the fluids from escaping the chamber 108.

The sump 114 is a location in the chamber 108 where the fluids may collect and be removed from the chamber 108 using the conduit 116. In an embodiment, the sump 114 may be occupied by the fluid permeable support 102 and/or the fluid permeable membrane 104, which allows for direct contact between the inlet 136 of the conduit 116 and the fluid permeable support 102 and/or the fluid permeable membrane 104.

Occupying the sump 114 with the fluid permeable support 102 and/or the fluid permeable membrane 104 may facilitate removal of the fluids from the chamber 108. For example, the fluids in the chamber 108 may include water. It is currently believed that hydrogen bonding between the water molecules of the fluids causes the fluids that are being suctioned into the conduit 116 to pull additional fluids towards the conduit 116. It is noted that any gaps between the fluid permeable support 102 and/or the fluid permeable membrane 104 and the conduit 116 (e.g., caused by the sump 114 not being occupied to the fluid permeable support 102 and/or the fluid permeable membrane 104) may break the hydrogen bonding between the water molecules. The lack of hydrogen bonding may prevent the pulling of the fluids into the conduit 116 when the sump 114 is not at or near or gravimetric low point of the chamber 108. Instead, the fluids flowing the fluid permeable support 102 and/or the fluid permeable membrane 104 may rely on wicking and gravity to move the fluids towards the sump 114 and the conduit 116, which may be slower or not possible if the sump 114 is at or near the gravimetric low point of the chamber 108. The hydrogen bonding between the water molecules and the direct contact between the fluid permeable support 102 and/or the fluid permeable membrane 104 and the inlet 136 of the conduit 116 decreases the need to position the inlet 136 of the conduit 116 at or near the expected gravimetric low point of the chamber 108.

In an embodiment, not shown, the fluid permeable support 102 and the fluid permeable membrane 104 do not completely occupy the sump 114. In other words, the sump 114 may include a substantially unoccupied reservoir. The fluids received by the fluid collection assembly 100 may be retained in the reservoir prior to removing the fluids from the chamber 108.

The fluid impermeable barrier 106 may extend from a proximal end region 132 to a distal end region 134. In an embodiment, the sump 114 may be located at or near the distal end region 134 since the distal end region 134 may be at or near a gravimetric low point of the chamber 108 during use. In such an embodiment, the fluid impermeable barrier 106 may extend from the proximal end region 132 to the opening 110 by a first distance and from the distal end region 134 to the opening 110 by a second distance. The second distance may be greater than the first distance which increases the volume of the sump 114 (e.g., to increase the quantity of fluids that may be stored in the sump 114) and inhibits fluids in the sump 114 from leaking from the chamber 108 through the opening 110.

The conduit 116 may be at least partially disposed in the chamber 108. The conduit 116 may be used to remove the fluids from the chamber 108. The conduit 116 (e.g., a conduit) includes an inlet 136 and an outlet positioned downstream from the inlet 136. The outlet may be operably coupled to a vacuum source, such as a vacuum pump for withdrawing fluid from the chamber 108 through the conduit 116. For example, the conduit 116 may extend into the fluid impermeable barrier 106 from the proximal end region 132 and may extend to the distal end region 134 to a point proximate to the reservoir therein such that the inlet 136 is in fluid communication with the reservoir. The conduit 116 fluidly couples the chamber 108 with the fluid storage container (not shown) or the vacuum source (not shown). In such an embodiment, at least one of the fluid permeable support 102 or the fluid permeable membrane 104 defines a bore (not labeled, occupied) through which the conduit 116 may extend. In an embodiment, the conduit 116 may extend generally parallel to the longitudinal axis 118 of the fluid collection assembly.

The conduit 116 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 116 may include silicon or latex. In some examples, the conduit 116 may include one or more portions that are resilient, such as having one or more of a diameter or wall thickness that allows the conduit to be flexible.

The inlet 136 at or near a location expected to be the gravimetrically low point of the chamber 108 (e.g., the sump 114) when worn by a patient which may enable the conduit 116 to receive more of the fluids than if inlet 136 was located elsewhere and reduce the likelihood of pooling (e.g., pooling of the fluids may cause microbe growth and foul odors). For instance, the fluids in the fluid permeable support 102 and the fluid permeable membrane 104 may flow in any direction due to capillary forces. However, the fluids may exhibit a preference to flow in the direction of gravity, especially when at least a portion of the fluid permeable membrane 118 and/or the fluid permeable support 102 is saturated with the fluids. Accordingly, one or more of the inlet 136 or the reservoir may be located in the fluid collection assembly 100 in a position expected to be the gravimetrically low point in the fluid collection assembly 100 when worn by a patient, such as the distal end region 134.

As described in more detail below, the conduit 116 is configured to be coupled to, and at least partially extend between, one or more of the fluid storage container (not shown) and the vacuum source (not shown). In an example, the conduit 116 is configured to be directly connected to the vacuum source (not shown). In such an example, the conduit 116 may extend from the fluid impermeable barrier 106 by at least one foot, at least two feet, at least three feet, or at least six feet. In another example, the conduit 116 is configured to be indirectly connected to at least one of the fluid storage container (not shown) and the vacuum source (not shown). In some examples, the conduit is secured to a patient's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

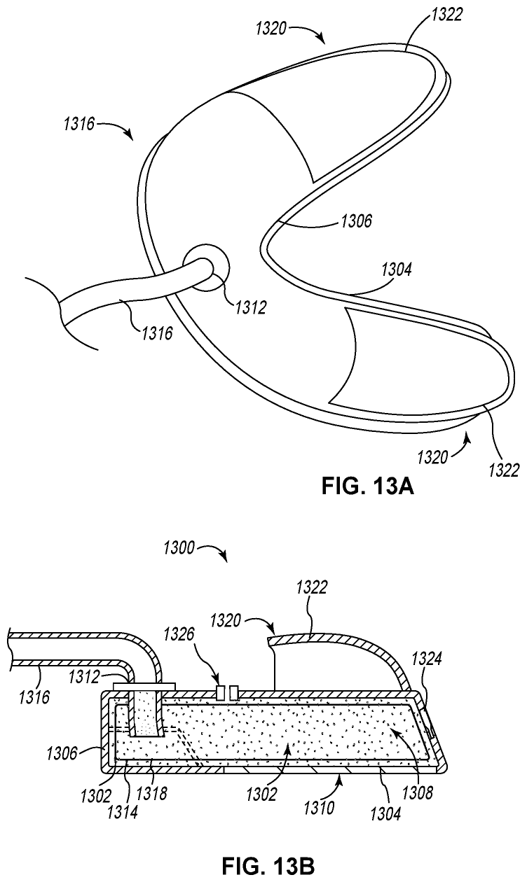

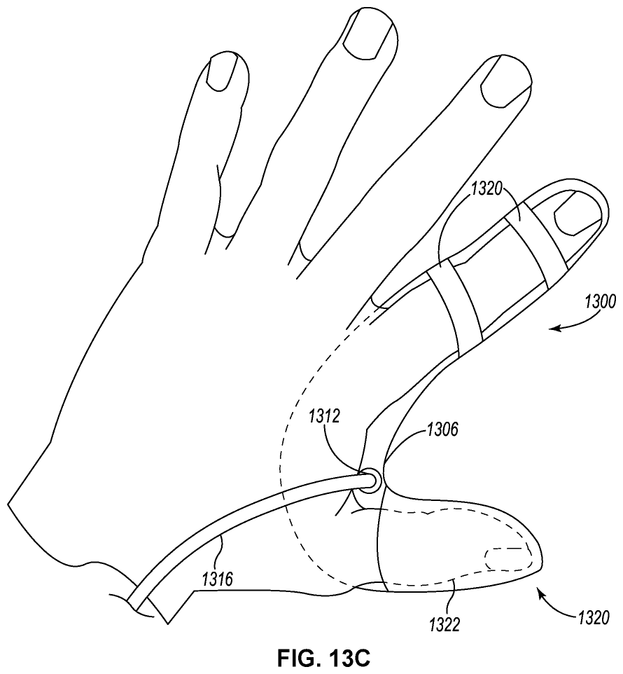

The inlet 136 and the outlet are configured to fluidly couple (e.g., directly or indirectly) the vacuum source (not shown) to the chamber 108 (e.g., the reservoir). As the vacuum source (FIG. 13) applies a vacuum/suction in the conduit 116, the fluids in the chamber 108 (e.g., at the distal end region such as in the reservoir) may be drawn into the inlet 136 and out of the fluid collection assembly 100 via the conduit 116. In some examples, the conduit may be frosted or opaque (e.g., black) to obscure visibility of the fluids therein.

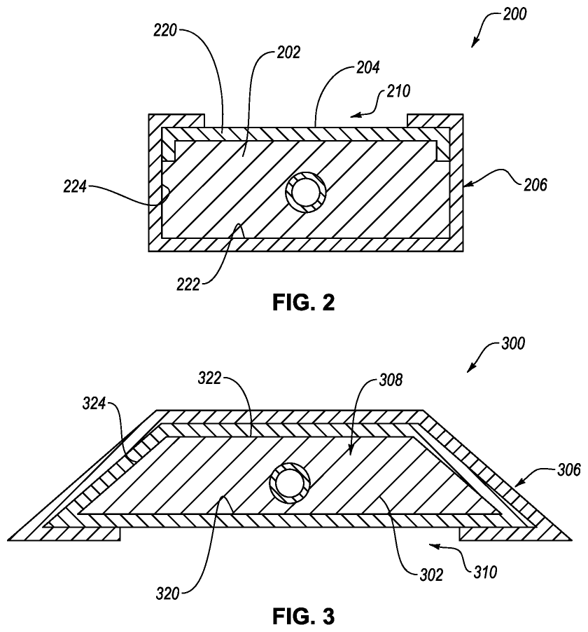

As previously discussed, the fluid permeable membrane of the fluid collection assemblies disclosed herein does not need to extend completely around the fluid permeable support. FIG. 2 is a cross-sectional schematic of a fluid collection assembly 200, according to an embodiment. Except as otherwise disclosed herein, the fluid collection assembly 200 is the same or substantially similar to any of the fluid collection assemblies disclosed herein. For example, the fluid collection assembly 200 includes a fluid permeable support 202, a fluid permeable membrane 204, and a fluid impermeable barrier 206 defining an opening 210.

The fluid permeable support 202 includes a first surface 220, a second surface 222 opposite the first surface 220, and one or more edges 224 extending from the first surface 220 to the second surface 222. The fluid permeable membrane 204 is disposed adjacent to at least a portion of the first surface 220 but does not extend completely around the fluid permeable support 202. For example, as illustrate, the fluid permeable membrane 204 may be disposed on all of the first surface 220 and a portion of the edges 224. To maintain the fluid permeable membrane 204 extending across the opening 210, the fluid permeable membrane 204 may be secured to the fluid permeable support 202 and/or the fluid impermeable barrier 206. The fluid permeable membrane 204 may be secured to the fluid permeable support 202 or the fluid impermeable barrier 206 using an adhesive, sewing, friction, or any other suitable attachment method.

The fluid collection assemblies illustrated in FIGS. 1A-2 is illustrated as exhibiting a generally rectangular cross-sectional shape. However, the fluid collection assemblies disclosed herein may exhibit any suitable shape (e.g., any suitable cross-sectional shape) so long as the fluid collection assemblies remains relatively thin. FIG. 3 is a cross-sectional schematic of a fluid collection assembly 300 exhibiting a generally non-rectangular cross-sectional shape, according to an embodiment. Except as otherwise disclosed herein, the fluid collection assembly 300 is the same or substantially similar to any of the fluid collection assemblies disclosed herein.

The fluid permeable support 302 exhibits a generally trapezoidal shape exhibiting a first surface 320, a second surface 322 opposite the first surface 320, and one or more edge 324 extending from the first surface 320 to the second surface 322. In an example, as illustrated, the first surface 320 may form the long side of the generally trapezoidal shape and the second surface 322 may form the short side of the generally trapezoidal shape. In such an example, the shape of the fluid permeable support 302 may maximize the size of the opening 310 defined by the fluid impermeable barrier 306 relative to a volume of the chamber 308. Further, such a shape may facilitate positioning the opening 310 adjacent to a female urethral opening since the edges 324 may better fit in the crevice formed between the vulva and the thighs better than if the fluid permeable support 302 exhibited a generally rectangular cross-sectional shape. In an example, the first surface 320 may form the short side of the generally trapezoidal shape and the second surface 322 may form the long side of the generally trapezoidal shape. In such an example, the chamber 308 may be able to have a larger volume of fluids present in the chamber 308 relative to the size of the opening 310.

It is noted that the fluid impermeable barrier 306 may also exhibit a generally trapezoidal shape that generally corresponds (e.g., is slightly larger) than the generally trapezoidal shape of the fluid permeable support 302.

It is noted that the fluid collection assemblies disclosed herein may exhibit a cross-sectional shape other than a generally rectangular cross-sectional shape (as shown in FIGS. 1A-2) or a generally trapezoidal cross-sectional shape (as shown in FIG. 3). In an example, the fluid collection assembly 100 may exhibit a semi-circular cross-sectional shape (e.g., a semi-cylindrical shape). In such an example, the flat side of the semi-circular cross-sectional shape of the fluid collection assembly 100 may be positioned adjacent to the skin in the individual. In an example, the fluid collection assemblies disclosed herein may exhibit a generally oval cross-sectional shape, a generally ellipsoidal cross-sectional shape, or other oblong cross-sectional shape.

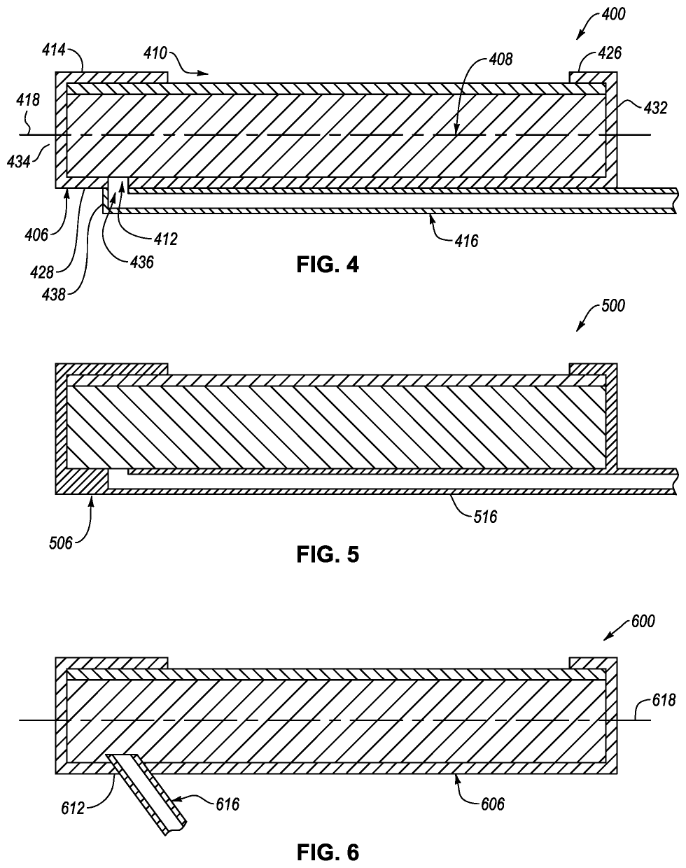

As illustrated in FIGS. 1B-3, the conduit may extend within the chamber, such as within a central portion of the chamber. However, the fluid collection assemblies may include a conduit that at least partially extends outside of the chamber due to the components and construction of the fluid collection assemblies disclosed herein. For example, FIGS. 4-6 are cross-sectional schematics of different fluid collection assemblies including a conduit that at least partially extends outside of the chamber, according to different embodiments. Except as otherwise disclosed herein, the fluid collection assemblies illustrated in FIGS. 4-6 may be the same or substantially similar to any of the fluid collection assemblies disclosed herein.

Referring to FIG. 4, the fluid collection assembly 400 includes a fluid impermeable barrier 406 extending from a proximal end region 432 to a distal end region 434. The fluid impermeable barrier 406 includes a front surface 426 that defines an opening 410 and a back surface 428 opposite the front surface 426. The fluid outlet 412 is formed in or near the back surface 428 at or near the sump 412. For example, the fluid outlet 412 may be formed in or near the back surface 428 at or near the distal end region 434 when the sump 414 is at or near the distal end region 434.

The conduit 416 may be attached to the fluid outlet 412 such that the conduit 416 is in fluid communication with the chamber 408. In an embodiment, as illustrated, the conduit 416 may extend from the fluid outlet 416 in a direction that is generally perpendicular to a longitudinal axis 418 of the fluid collection assembly 400. In other words, at least a portion of the conduit 416 may extend along the back surface 428 of the fluid impermeable barrier 406. The conduit 416 may be attached to the back surface 428, for example using an adhesive, to maintain the conduit 416 against the back surface 428 of the fluid impermeable barrier 406. Extending the conduit 416 along the back surface 428 allows the conduit 416 to extends from the fluid collection assembly 400 from or near the proximal end region 432, similar to the conduit 116 illustrated in FIGS. 1A-1C. Extending the conduit 416 from or near the proximal end region 432 allows the conduit 416 to extend along a surface (e.g., skin) and allows one or more elements to be disposed on top of the fluid collection assembly 400 without interfering with the conduit 416 and vice versa. For example, extending the conduit 416 from or near the proximal end region 432 may allow underwear to be disposed on top of the fluid collection assembly 400 when the fluid collection assembly 400 is disposed on top of a urethral opening or a bandage to be disposed on top of the fluid collection assembly 400 when the fluid collection assembly 400 is disposed on a wound. Further, allowing the conduit 416 to extend along the back surface 428 may prevent the need to form a bore in at least one of the fluid permeable support 402 or the fluid permeable membrane 402 thereby simplifying the manufacturing of the fluid collection assembly 400 and increasing the volume of fluids that may be present in the chamber 408.

In an embodiment, as illustrated, the conduit 416 does not extend into the chamber 408 which may prevent the need to form a bore in at least one of the fluid permeable support 402 or the fluid permeable membrane 402 thereby simplifying the manufacturing of the fluid collection assembly 400 and increasing the quantity of fluids that may be stored in the chamber 408. In such an embodiment, the inlet 436 may not be formed or completely defined by at the terminal end 438 of the walls the form the conduit 416. Instead, the inlet 436 may be proximate to or only partially formed by the terminal end 438 of the conduit 416 which allows the inlet 436 to receive fluids from a side thereof and prevent the need to form a bend in the conduit 416. In an embodiment, the conduit 416 may extend from the fluid outlet 412 into the chamber 408.

Referring to FIG. 5, the fluid collection assembly 500 includes a fluid impermeable barrier 506 and a conduit 516. In an embodiment, an embodiment, the fluid impermeable barrier 506 may form at least a portion of the conduit 516 (e.g., the fluid impermeable barrier 506 and the conduit 516 are integrally formed together). In such an embodiment, the fluid impermeable barrier 506 may defined a hollow passageway 540 that is configured to remove one or more fluids from the chamber 508. Forming at least a portion of the conduit 516 with the fluid impermeable barrier 506 may decrease the number of components that form the fluid collection assembly 500. In an embodiment, not shown, the fluid collection assembly 500 may include a conduit disposed in the hollow passageway 540. In such an embodiment, the hollow passageway 540 may secure the conduit to the fluid impermeable barrier 506.

Referring to FIG. 6, the fluid collection assembly 600 includes a fluid impermeable barrier 606 and a conduit 616. Unlike the conduits illustrated in FIGS. 4 and 5, the conduit 616 does not extend along a surface (e.g., back surface 628) of the fluid impermeable barrier 606 or extends generally parallel to a longitudinal axis 618 of the fluid collection assembly 600. Instead, the conduit 616 may extend away from the fluid outlet 612 in a direction that is perpendicular to or obliquely angled relative to a longitudinal axis 618 of the fluid collection assembly 600. In such an embodiment, the conduit 616 may extend away from the back surface 628 of the fluid impermeable barrier 606. It is noted that the conduit 616 may extend from other surfaces of the fluid impermeable barrier 606.

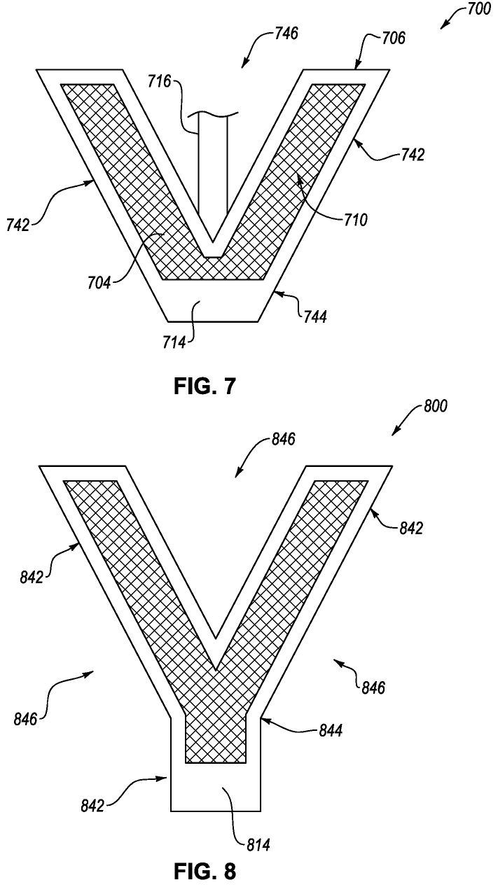

As previously discussed, the opening of the fluid collection assemblies disclosed herein, and in particular the openings thereof, may exhibit a limited range of lengths and widths otherwise the suction provided to the chamber of the fluid collection assemblies becomes too dispersed to effectively remove fluids from the fluid collection assembly. The limited lengths and widths of the opening of the fluid collection assemblies disclosed herein prevent the fluid collection assemblies from being a pad (e.g., a collection assembly including an opening exhibiting a length, width, or area that causes too much dissipation of the suction). Preventing the fluid collection assembly from being a pad may limit the area from which the fluid collection assembly may receive fluids. One solution is to cause the fluid collection assemblies disclosed herein to exhibit a shape including one or more in-plane bends therein. The in-plane bend(s) allows the fluid collection assemblies to define gaps between portions thereof. The fluid collection assemblies may receive bodily fluids from fluid sources adjacent to the fluid collection assemblies or, possible, from a fluid source disposed in the gap. As such, the in-plane bends allow the fluid collection assemblies disclosed herein to collect fluids from a large surface area while allowing the opening of the fluid collection assemblies to not exhibit a width, length, and/or surface area that causes the suction source to dissipate. Further, the in-plane bends allows for improved movement of the fluids in the porous materials thereof. FIGS. 7-11 are top plan view of fluid collection assemblies exhibiting at least one in-plane bend, according to different embodiments. Except as otherwise disclosed herein, the fluid collection assemblies illustrated in FIGS. 7-11 may be the same or substantially similar to any of the fluid collection assemblies disclosed herein. For example, the fluid collection assemblies illustrated in FIGS. 7-11 may include a fluid permeable support (not shown, obscured), a fluid permeable membrane, and a fluid impermeable barrier defining a chamber (not shown, occupied), an opening, a fluid outlet, and a sump.

Referring to FIG. 7, the fluid collection assembly 700 includes a plurality of branches 742 (e.g., a plurality of elongated branches) and a common area 744 extending between at least two adjacent branches 742. The branches 742 may extend from the common area 744 in different directions. In other words, the common area 744 forms a bridge and an in-plane bend between the branches 742. Thus, the common area 744 allows the fluid collection assembly 700 to exhibit a relatively thin shape that is bent. The branches 742 may define a gap 746 therebetween. The fluid impermeable barrier 706 may form the portion of the branches 742 that define the gap (e.g., cover the edges of the fluid permeable support that form the gap 746) to prevent the suction from being dispersed into the gap 746 and to prevent the opening 710 from one branch 742 contacting the opening 710 from another branch 742 which may effectively increase the collectively width of the two portions of the opening 710.

In an embodiment, as illustrated, the fluid collection assembly 700 includes two branches 742. The two branches 742 extend from the common area 744 at an oblique or perpendicular angle relative to each other thereby allowing the fluid collection assembly 700 to exhibit a shape that is different than the fluid collection assembly illustrated in FIG. 1A. The two branches 742 and the common area 744 cause the fluid collection assembly 700 to exhibit a generally V-like shape. The generally V-like shape may allow the fluid collection assembly 700 to receive fluids from a large surface area, similar to a pad. For example, the generally V-like shape may allow the fluid collection assembly 700 to receive fluids from a fluid source adjacent to the opening 710 and probably receive fluids from a fluid source positioned adjacent to the gap 746.

In an embodiment, the common area 744 may form the sump 714 of the fluid collection assembly 700 since the fluids received by each of the branches 742 may flow into the common area 744 and the common area 744 may be centrally located. Further, the common area 744 may be the gravimetric low point of the chamber of the fluid collection assembly 700. The fluid impermeable barrier 706 may extend inwardly more from an edge of the common area 744 than an edge of the branches 742 to increase the volume of the sump 714 and the quantity of fluids that may be present in the sump 714. In an embodiment, the fluid outlet (not shown, obscured) may be formed in the common area 742 and the conduit 716 may be connected to the fluid outlet. In an embodiment, the sump 714 may be spaced from the common area 744, for example, when the common area 744 is not the gravimetric low point of the chamber.

It is noted that at least one of the fluid permeable support, the fluid permeable membrane 704, or the fluid impermeable barrier 706 may exhibit a shape that generally corresponds to the shape of the fluid collection assembly 700, as a whole. For example, at least one of the fluid permeable support, the fluid permeable membrane 704, or the fluid impermeable barrier 706 may include two branches extending from a common area. It is noted that the opening 710 may also exhibit a shape that generally corresponds to the shape of the fluid collection assembly 700 as a whole which allows the opening 710 to receive fluids from a large surface area.

The fluid collection assemblies disclosed herein may include 2 or more branches (e.g., 3, 4, 5, 6, 7, 8, 9, or 10 or more branches) extending from a single common area. For example, referring to FIG. 8, the fluid collection assembly 800 includes three branches 842 extending from a common area 844. The three branches 842 and the common area 844 may cause the fluid collection assembly 800 to exhibit a generally Y-like shape. The branches 842 may form gaps 846 between each of the branches 742. The fluid collection assembly 800 may be configured to receive fluids from a fluid source adjacent to the opening 810 and/or a fluid source positioned adjacent to the gaps 846.

In an embodiment, as illustrated, the sump 814 may be located at the terminal end of one of the branches 842. For example, the sump 814 may be located at the terminal end of one of the branches 842 when that particular branch 842 forms the expected gravimetric low point of the chamber of the fluid collection assembly 800. The fluid impermeable barrier 806 may extend inwardly more from the terminal end the branch 842 than another edge to increase the volume of the sump 814 and the quantity of fluids that may be stored in the sump 814. In an embodiment, the sump 814 may be at the common area 844 since the common area may be more centrally located than the terminal end of a branch 842.

It is noted that the fluid collection assemblies disclosed herein may include more than three branches, such as 4, 5, 6, 7, 8, 9, 10 or greater than 10 branches.

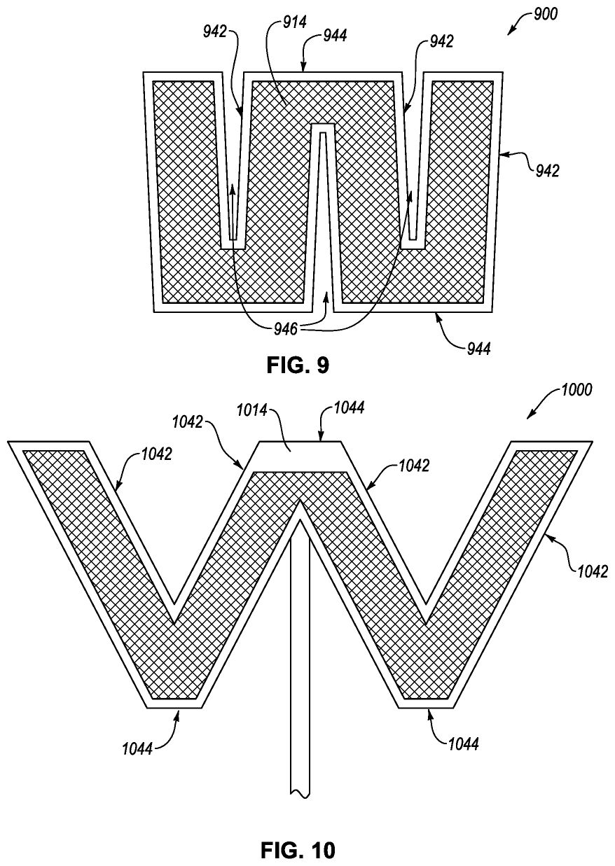

The fluid collection assemblies disclosed herein may include a plurality of common areas instead of the single common area. For example, referring to FIG. 9, the fluid collection assembly 900 includes a plurality of branches 942 and a plurality of common areas 944. Each of the common areas 944 includes at least two branches 942 extending therefrom. At least some of the branches 942 extend between different common areas 944. In an embodiment, as illustrated, the branches 942 may extend from the common areas 944 in a direction that is generally parallel to each other. The plurality of common areas 944 allows the fluid collection assembly 900 to exhibit a relatively thin shape that is bent to form a zig-zag shape. The zig-zag shape along with the branches 942 extending generally in-plane to each other allows the fluid collection assembly 900 to generally exhibit a pad-like shape except for the gaps 946 between the branches 942. The gaps 946 limits the width of the opening 910 at any given point thereby preventing the dissipation of the suction as would occur in a pad (e.g., a collection assembly that does not include the gaps 946) that is substantially similar to the fluid collection assembly 900. In an embodiment, the sump 914 may be located at a common area 944 (e.g., a centrally located common area 944), at a terminal end of one of the branches 942, or at any location therebetween.

Referring to FIG. 10, the fluid collection assembly 1000 includes a plurality of branches 1042 and a plurality of common areas 1044. Similar to the fluid collection assembly 900 of FIG. 9, the common areas 1044 includes at least two branches 1042 extending therefrom and at least some of the branches 1042 extend between different common areas 1044 such that the fluid collection assembly 1000 to exhibit a generally zig-zag shape. However, at least some of the branches 1042 may extend from the common areas 1044 in a direction that is generally obliquely angled relative to each other. The obliquely angled branches 1042 causes the fluid collection assembly 1000 to be spread out more on a surface than the fluid collection assembly 900 thereby allowing the fluid collection assembly 1000 to potentially receive fluids from a larger surface area. In an embodiment, the sump 1014 may be located at a common area 1044 (e.g., a centrally located common area 1044), at a terminal end of one of the branches 1042, or at any location therebetween.

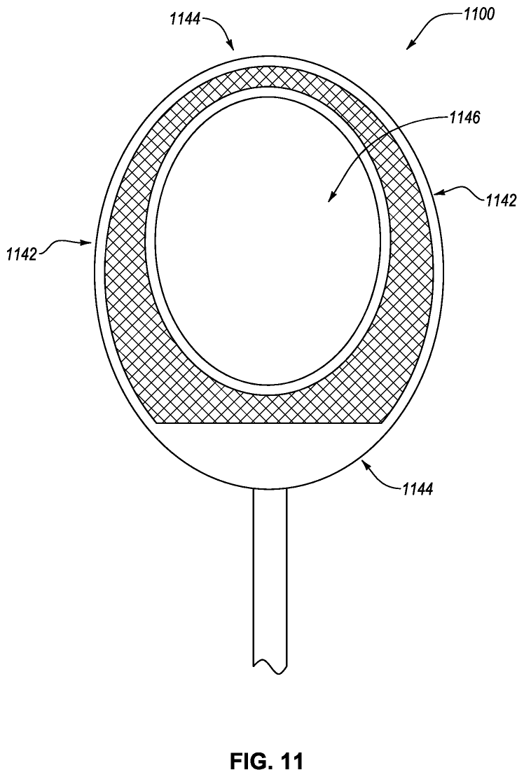

Referring to FIG. 11, the fluid collection assembly 1100 includes a plurality of relatively thin branches 1142 and a plurality of common areas 1144. The plurality of branches 1142 includes two branches 1142 that extend between the same two common areas 1144 and define a gap 1146 therebetween. In other words, the two branches 1142 and the two common areas 1144 form a generally hollow annular shape (e.g., a generally hollow circular, a generally hollow oval, or generally hollow ellipsoid shape). In an embodiment, the fluid collection assembly 1100 may be positioned with the fluid source adjacent to the opening 1110 or within the gap 1146 such that the fluid collection assembly 1100 may receive fluids from the fluid source regardless of the direction that the fluids flow from the fluid source.

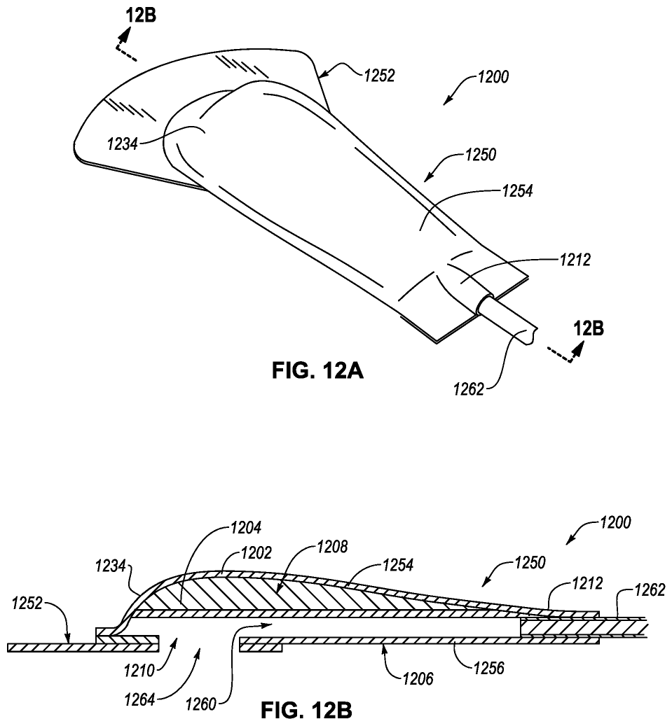

In the embodiments discussed above, the fluid impermeable barrier exhibits a shape that generally corresponds to the shape of the fluid permeable support. However, in some embodiments, the fluid impermeable barrier does not exhibit a shape that generally corresponds to the shape of the fluid permeable support. For example, FIG. 12A is an isometric view of a fluid collection assembly 1200 that includes a fluid impermeable barrier 1206 that does not exhibit a shape that generally corresponds to the shape of the fluid permeable membrane 1202, according to an embodiment. FIG. 12B is a cross-sectional schematic of the fluid collection assembly 1200 taken along plane 12B-12B. Except as otherwise disclosed herein, the fluid collection assembly 1200 may be the same or substantially similar to any of the fluid collection assemblies disclosed herein.

The fluid collection assembly 1200 is an example of a male fluid collection assembly though, in some embodiments, the fluid collection assembly 1200 may be used to receive bodily fluids from a female urethral opening. The fluid collection assembly 1200 includes a sheath 1250 and a base 1252. The base 1252 is configured to be attached (e.g., permanently attached to or configured to be permanently attached) to the sheath 1250. The base 1252 is also configured to be attached to the region about the urethral opening (e.g., penis) of the individual.

The sheath 1250 includes a fluid impermeable barrier 1206 that is at least partially formed from a first panel 1254 and a second panel 1256. The first panel 1254 and the second panel 1256 may be attached or integrally formed together (e.g., exhibits single piece construction). In an embodiment, as illustrated, the first panel 1254 and the second panel 1256 are distinct sheets. The fluid impermeable barrier 1206 also defines a chamber 1208 between the first panel 1254 and the second panel 1256, an opening 1210 at a proximal end region 1564 of the sheath 1250, and a fluid outlet 1212 at a distal end region 1562 of the sheath 1250. The sheath 1250 also includes a fluid permeable support 1202 and a fluid permeable membrane 1204 disposed in the chamber 1208.

The inner surface(s) of the fluid impermeable barrier 1206 (e.g., inner surfaces of the first and second panels 1254, 1256 at least partially defines the chamber 1208 within the fluid collection assembly 1200. The fluid impermeable barrier 1206 temporarily stores the bodily fluids in the chamber 1208. The fluid impermeable barrier 1206 may be formed from any of the fluid impermeable materials disclosed herein. As such, the fluid impermeable barrier 1206 substantially prevents the bodily fluids from passing through the fluid impermeable barrier 1206.

In an embodiment, at least one of the first panel 1254 or the second panel 1256 is formed from an at least partially transparent fluid impermeable material, such as polyethylene, polypropylene, polycarbonate, or polyvinyl chloride. Forming at least one of the first panel 1254 or the second panel 1256 from an at least partially transparent fluid impermeable material allows a person (e.g., medical practitioner) to examine the penis. In some embodiments, both the first panel 1254 and the second panel 1256 are formed from at least partially transparent fluid impermeable material. Selecting at least one of the first panel 1254 or the second panel 1256 to be formed from an at least partially transparent impermeable material allows the penis to be examined without detaching the entire fluid collection assembly 1200 from the region about the penis. For example, the chamber 1208 may include a penis receiving area 1260 that is configured to receive the penis of the individual when the penis extends into the chamber 1208. The penis receiving area 1260 may be defined by at least the fluid permeable membrane 1204 and at least a portion of the at least partially transparent material of the first panel 1254 and/or the second panel 1256. In other words, the fluid permeable membrane 1204 is positioned in the chamber 1208 such that the fluid permeable membrane 1204 is not positioned between the penis and at least a portion of the transparent portion of the first panel 1254 and/or second panel 1256 when the penis is inserted into the chamber 1208 through the opening 1210. The fluid permeable membrane 1204 is generally not transparent and, thus, the portion of the at least partially transparent material of the first panel 1254 and/or the second panel 1256 that defines the penis receiving area 1260 forms a window which allows the person to view into the penis receiving area 1260 and examine the penis.

The opening 1210 defined by the fluid impermeable barrier 1206 provides an ingress route for bodily fluids to enter the chamber 1208 when the penis is a buried penis and allow the penis to enter the chamber 1208 (e.g., the penis receiving area 1260) when the penis is not buried. The opening 1210 may be defined by the fluid impermeable barrier 1206 (e.g., an inner edge of the fluid impermeable barrier 1206). For example, the opening 1210 is formed in and extends through the fluid impermeable barrier 1206 thereby enabling bodily fluids to enter the chamber 1208 from outside of the fluid collection assembly 1200.

The fluid impermeable barrier 1206 defines the fluid outlet 1212 sized to receive the conduit 1262. The conduit 1262 may be at least partially disposed in the chamber 1208 or otherwise in fluid communication with the chamber 1208 through the fluid outlet 1212. The fluid outlet 1212 may be sized and shaped to form an at least substantially fluid tight seal against the conduit 1262 thereby substantially preventing the bodily fluids from escaping the chamber 1208. In an embodiment, the fluid outlet 1212 may be formed from a portion of the first panel 1254 and the second panel 1256 that are not attached or integrally formed together. In such an embodiment, the fluid impermeable barrier 1206 may not include a cap exhibiting a rigidity that is greater than the portions of the fluid impermeable barrier 1206 thereabout which may facilitate manufacturing of the fluid collection assembly 1200 may decreasing the number of parts that are used to form the fluid collection assembly 1200 and may decrease the time required to manufacture the fluid collection assembly 1200. The lack of the cap may make securing the conduit 1262 to the fluid outlet 1212 using interference fit to be difficult though, it is noted, attaching the conduit 1262 to the fluid outlet 1212 may still be possible. As such, the conduit 1262 may be attached to the fluid outlet 1212 (e.g., to the first and second panels 1254, 1256) using an adhesive, a weld, or otherwise bonding the fluid outlet 1212 to the fluid outlet 1212. Attaching the conduit 1262 to the fluid outlet 1212 may prevent leaks and may prevent the conduit 1262 from inadvertently becoming detached from the fluid outlet 1212. In an example, the conduit 1262 may be attached to the fluid outlet 1212 in the same manufacturing step that attaches the first and second panels 1254, 1256 together.

The fluid collection assembly 1200 also includes the fluid permeable support 1202 and the fluid permeable membrane 1204 disposed in the chamber 1208. The fluid permeable support 1202 and the fluid permeable membrane 1204 may direct the bodily fluids to one or more selected regions of the chamber 1208, such as away from the penis and towards the fluid outlet 1212. The fluid permeable support 1202 and the fluid permeable membrane 1204 may be formed from any of the fluid permeable membranes and fluid permeable supports, respectively, disclosed herein.

In an embodiment, the fluid permeable support 1202 and the fluid permeable membrane 1204 may be a sheet. Forming the fluid permeable support 1202 and the fluid permeable membrane 1204 as a sheet may facilitate the manufacturing of the fluid collection assembly 1200. For example, forming the fluid permeable support 1202 and the fluid permeable membrane 1204 as a sheet allows the first panel 1254, the second panel 1256, the fluid permeable support 1202, and the fluid permeable membrane 1204 to each be sheets. During the manufacturing of the fluid collection assembly 1200, the first panel 1254, the second panel 1256, the fluid permeable support 1202, and the fluid permeable membrane 1204 may be stacked and then attached to each other in the same manufacturing step. For instance, the fluid permeable support 1202 and the fluid permeable membrane 1204 may exhibit a shape that is the same size or, more preferably, slightly smaller than the size of the first panel 1254 and the second panel 1256. As such, attaching the first panel 1254 and the second panel 1256 together along the outer edges thereof may also attach the fluid permeable support 1202 and the fluid permeable membrane 1204 to the first panel 1254 and the second panel 1256. The fluid permeable support 1202 and the fluid permeable membrane 1204 may be slightly smaller than the first panel 1254 and the second panel 1256 such that the first panel 1254 and/or the second panel 1256 extend around the fluid permeable support 1202. Also, attaching the fluid permeable support 1202 and the fluid permeable membrane 1204 to the first panel 1254 and/or the second panel 1256 may prevent the fluid permeable support 1202 and the fluid permeable membrane 1204 from significantly moving in the chamber 1208, such as preventing the fluid permeable support 1202 and the fluid permeable membrane 1204 from bunching together near the fluid outlet 1212. In an example, the fluid permeable support 1202 and the fluid permeable membrane 1204 may be attached to the first panel 1254 or the second panel 1256 (e.g., via an adhesive) before or after attaching the first panel 1254 to the second panel 1256. In an example, the fluid permeable support 1202 and the fluid permeable membrane 1204 may merely be disposed in the chamber 1208 without attaching the fluid permeable support 1202 and the fluid permeable membrane 1204 to at least one of the first panel 1254 or the second panel 1256. In an embodiment, the fluid permeable support 1202 and the fluid permeable membrane 1204 may exhibit shapes other than a sheet, such as a hollow generally cylindrical shape.

In an embodiment, the fluid impermeable barrier 1206 does not exhibit a shape that generally corresponds to the shape of the fluid permeable membrane 1204 and the fluid permeable support 1022. In such an example, the fluid impermeable barrier 1206 is a bag, such as a bag exhibiting a generally rectangular shape. The fluid permeable support 1202 and the fluid permeable membrane 1204 and include a plurality of branches extending from a common area. The plurality of branches may define a gap 1246 therebetween.

Generally, the sheath 1250 is substantially flat when the penis is not in the penis receiving area 1260 and the sheath 1250 is resting on a flat surface. The sheath 1250 is substantially flat because the fluid impermeable barrier 1206 is formed from the first panel 1254 and the second panel 1256 instead of a generally tubular fluid impermeable barrier. Further, as previously discussed, the fluid permeable support 1202 and the fluid permeable membrane 1204 may be a sheet, which also causes the sheath 1250 to be substantially flat. The sheath 1250 may also be substantially flat because the fluid collection assembly 1200 may not include relatively rigid rings or caps that exhibit a rigidity that is greater than the portions of the fluid impermeable barrier 1206 thereabout since such rings and caps may inhibit the sheath 1250 being substantially flat. It is noted that the sheath 1250 is described as being substantially flat because at least one of the fluid permeable support 1202 and the fluid permeable membrane 1204 may cause a slight bulge to form in the sheath 1250 depending on the thickness of the fluid permeable support 1202, the fluid permeable membrane 1204, the fluid outlet 1212 and/or conduit 1262 may cause a bulge thereabout, or the base 1252 may pull on portions of the sheath 1250 thereabout. It is also noted that the sheath 1250 may also be compliant and, as such, the sheath 1250 may not be substantially flat during use since, during use, the sheath 1250 may rest on a non-flat surface (e.g., may rest on the testicles, the perineum, and/or between the thighs) and the sheath 1250 may conform to the surface of these shapes.

The ability of the sheath 1250 to be substantially flat when the penis is not in the penis receiving area 1260 and the sheath 1250 is resting on a flat surface allows the fluid collection assembly 1200 to be used with a buried and a non-buried penis. For example, when the fluid collection assembly 1200 is being used with a buried penis, the penis does not extend into the penis receiving area 1260 which causes the sheath 1250 to lie relatively flat across the aperture 1264 of the base 1252. When the sheath 1250 lies relatively flat across the aperture 1264, the fluid permeable membrane 1204 may extend across the opening 1210 and the aperature 1264 and is in close proximity to the buried penis. As such, the fluid permeable support 1202 and the fluid permeable membrane 1204 prevents or inhibits pooling of bodily fluids discharged from the buried penis against the skin of the individual since the fluid permeable support 1202 and the fluid permeable membrane 1204 will receive and remove at least a significant portion of the bodily fluids that would otherwise pool against the skin of the individual. Thus, the skin of the individual remains dry thereby improving comfort of using the fluid collection assembly 1200 and preventing skin degradation. However, unlike other conventional fluid collection assemblies that are configured to be used with buried penises, the fluid collection assembly 1200 may still be used with a non-buried penis since the non-buried penis can still be received into the penis receiving area 1260, even when the penis is fully erect. Additionally, the ability of the sheath 1250 to be substantially flat allows the fluid collection assembly 1200 to be used more discretely than if the sheath 1250 was not substantially flat thereby avoiding possibly embarrassing scenarios.

When the sheath 1250 is substantially flat, the fluid permeable support 1202 and the fluid permeable membrane 1204 occupies substantially all of the chamber 1208 and the penis receiving area 1260 is collapsed (shown as being non-collapsed in FIG. 12B for illustrative purposes to show the penis receiving area 1260). In other words, the sheath 1250 may not define a region that is constantly unoccupied by the fluid permeable support 1202 and the fluid permeable membrane 1204. When the fluid permeable support 1202 and the fluid permeable membrane 1204 occupies substantially all of the chamber 1208, the bodily fluids discharged into the chamber 1208 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the fluid collection assembly 1200 includes a base 1252 that is configured to be attached to the sheath 1250. For example, the base 1252 is configured to be permanently attached to the sheath 1250. The base 1252 is configured to be permanently attached to the sheath 1250 when, for example, when the fluid collection assembly 1200 is provided with the base 1252 permanently attached to the sheath 1250 or the base 1252 is provided without being permanently attached to the sheath 1250 but is configured to be permanently attached to the sheath 1250 at some point in the future. Permanently attached means that the sheath 1250 cannot be detached from the base 1252 without damaging at least one of the sheath 1250 or the base 1252, using a blade to separate the sheath 1250 from the base 1252, and/or using chemicals to dissolve the adhesive that attaches the sheath 1250 from the base 1252. The base 1252 may be permanently attached to the sheath 1250 using an adhesive, sewing, heat sealing, RF welding, or US welding. In an embodiment, the base 1252 is configured to be reversibly attached to the sheath 1250. In an embodiment, the base 1252 is integrally formed with the sheath 1250.

The base 1252 includes an aperture 1264. The base 1252 is permanently attached to the distal end region 1234 of the sheath 1250 such that the aperture 1264 is aligned with the opening 1210.

The base 1252 is sized, shaped, and made of a material to be coupled to the skin that surrounds the penis (e.g., mons pubis, thighs, testicles, and/or perineum) and have the penis disposed therethrough. For example, the base 1252 may define an aperture 1264 configured to have the penis positioned therethrough. In an example, the base 1252 may exhibit the general shape or contours of the skin surface that the base 1252 is configured to be coupled with. The base 1252 may be flexible, thereby allowing the base 1252 to conform to any shape of the skin surface and mitigate the base 1252 pulling the on skin surface. The base 1252 may extend laterally past the sheath 1250 thereby increasing the surface area of the skin of the individual to which the fluid collection assembly 1200 may be attached compared to a substantially similar fluid collection assembly 1200 that did not include a base.

Further examples of the sheath and the base are disclosed in U.S. Provisional Patent Application No. 63/067,542 filed on Aug. 19, 2020, the disclosure of which is incorporated herein, in its entirety, by this reference.

FIG. 13A is an isometric view of a fluid collection assembly 1300, according to an embodiment. FIG. 13B is a cross-sectional schematic of the fluid collection assembly 1300. The fluid collection assembly 1300 includes a fluid permeable support 1302 within a fluid impermeable barrier 1306, and a fluid permeable membrane 1304. The fluid impermeable barrier 1306 may define at least a chamber 1308, an opening 1310, a fluid outlet 1312, and a sump 1314. The fluid permeable support 1302 and the fluid permeable membrane 1304 may be disposed in the chamber 1308 such that the fluid permeable membrane 1304 extends across the opening 1310. The fluid collection assembly 1300 also includes a conduit 1316 that is in fluid communication with the chamber 1308 such that one or more fluids received into the chamber 1308 may be removed therefrom using the conduit 1316. The fluid permeable support 1302 exhibits a relatively thin shape, such as any of the relatively thin shapes disclosed herein. The fluid permeable support 1302 is configured to support the fluid permeable membrane 1304. The fluid permeable membrane 1304 may be composed to wick the fluids away from the opening 1306, thereby pulling the fluids into the chamber and preventing the fluids and the vacuum from escaping the chamber 1308.

In some embodiments, the conduit 1316 includes an inlet 1318 disposed in or adjacent to the sump 1314. The conduit 1316 may be at least partially disposed in the chamber 1308. The conduit 1316 may be used to remove the fluids from the chamber 1308. The conduit 1316 includes the inlet 1318 and an outlet positioned downstream from the inlet 1318. The outlet may be operably coupled to a vacuum source. The conduit 1316 fluidly couples the chamber 1308 with the fluid storage container (not shown) or the vacuum source (not shown). The sump 1314 is a location in the chamber 1308 where the fluids may collect and be removed from the chamber 1308 using the conduit 1316. In an embodiment, the sump 1314 may be occupied by the fluid permeable support 1302 and/or the fluid permeable membrane 1304, which allows for direct contact between the inlet 1318 of the conduit 1316 and the fluid permeable support 1302 and/or the fluid permeable membrane 1304. The conduit 1316 may couple to the fluid outlet or extend through the fluid impermeable barrier 1306 on a surface opposite the opening 1310 so that the conduit 1316 does not interfere with the opening 1310.

Occupying the sump 1314 with the fluid permeable support 1302 and/or the fluid permeable membrane 1304 may facilitate removal of the fluids from the chamber 1308. The direct contact between the fluid permeable support 1302 and/or the fluid permeable membrane 1304 and the inlet 1318 of the conduit 1316 decreases the need to position the inlet 1318 of the conduit 1316 at or near the expected gravimetric low point of the chamber 1308. In some embodiments, the fluid permeable support 1302 can extend into the inlet 1318 of the conduit 1316.

As noted above, the fluid collection assembly 1300 further includes the fluid impermeable barrier 1306. The fluid impermeable barrier 1306 substantially prevents the fluids from passing through the fluid impermeable barrier 1306. In an example, the fluid impermeable barrier 1306 may be air permeable and fluid impermeable. The fluid impermeable barrier 1306 may include a distal end region 1332 and a proximal end region 1334. In an embodiment, the fluid impermeable barrier 1306 may be formed from a flexible material, such as silicone, which allows the fluid impermeable barrier 1306 to be a shape that conforms the anatomy of the individual. The fluid impermeable barrier 1306 exhibits a shape that generally corresponds to a shape of the fluid permeable support 1302. As such, in the illustrated embodiment, the fluid impermeable barrier 1306 may exhibit a shape that can be used during surgery to wick away blood, irrigation fluid, liquid stool, amniotic fluid, bowel contents, etc. during surgery. In some embodiments, the fluid collection assembly 1300 may be shaped such that a surgeon may place the fluid collection assembly 1300 into a surgical field and the surgeon may blot fluid with the fluid collection assembly 1300 and the fluid is drawn into the fluid collection assembly 1300 through the opening 1310.

The opening 1310 provides an ingress route for the fluids to enter the chamber 1308. The opening 1310 may be defined by the fluid impermeable barrier 1306 such as by an inner edge of the fluid impermeable barrier 1306. For example, the opening 1310 is formed in and extends through the fluid impermeable barrier 1306 thereby enabling fluid(s) to enter the chamber 1308 from outside of the fluid collection assembly 1300. The opening 110 may be located and shaped to be positioned adjacent to a fluid source. As such, the fluid collection assembly 1300 may be used in surgery in place of a Yankauer aspirator and/or in place of surgical sponges. The fluid collection assembly 1300 may be positioned proximate to the fluid source and fluid may enter the chamber of the fluid collection assembly 1300 via the opening 1310. The fluid collection assembly 1300 is configured to receive the fluids into the chamber 1308 via the opening 1310.

The fluid collection assembly 1300 may further include at least one grip 1320. The grip 1320 may be coupled to the fluid impermeable barrier 1306. In some embodiments, the at least one grip 1320 may be coupled to an outer surface of the fluid impermeable barrier 1306. In some embodiments, the grip 1320 may be coupled to a surface of the fluid impermeable barrier 1306 opposite the opening 1310. The fluid collection assembly 1300 having a grip 1320 can be controlled by the surgeon or other user by being able to control the fluid removal. The surgeon or nurse can insert a digit into the grip 1320 and use the grip 1320 to position the assembly while performing surgery. When a finger is inserted into the grip 1320 during surgery, the user can use the grip to position the opening 1310 over a surgical site to remove blood and other fluid from the site.

Referring to FIGS. 13A-16, the grip 1320 can be held onto a finger of a user by various means. In some embodiments, the grip 1320 may be configured to be worn on a single digit or two digits of a hand. When worn on the 4th or 5th digit or both the 4th and 5th fingers of a surgeon or nurses hand, the surgeon, assistant, or nurse can blot fluid within the surgical opening where it is needed and still have use of the more primary fingers (the 1st_3rd digits, as labeled on FIGS. 13C, FIG. 14 and FIG. 16, respectively) for holding surgical instruments. The fluid collection assembly 1300 may also minimize the risk of needle stick injuries by minimizing the number of fingers and/or hands in the surgical field.

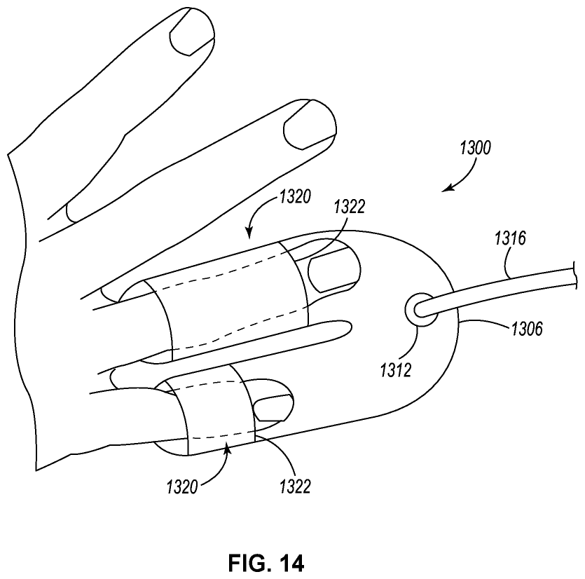

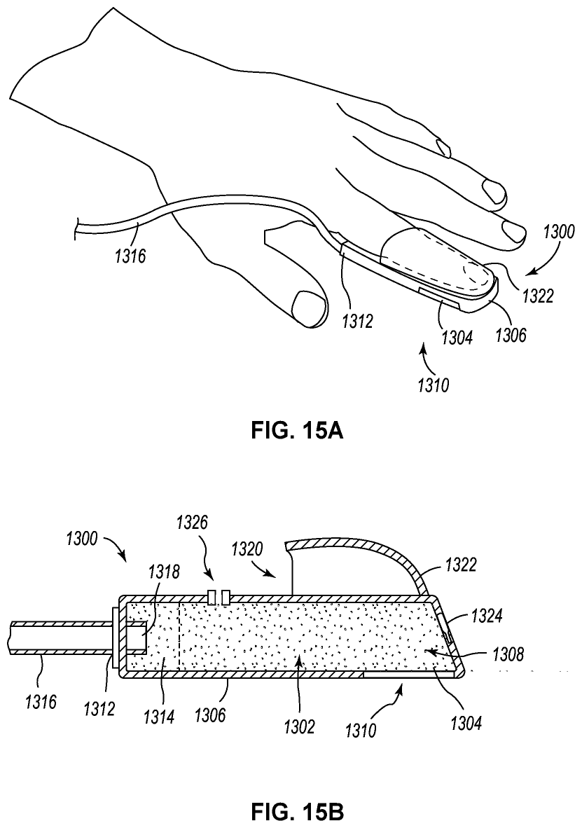

In some embodiments, as shown in FIGS. 13A and FIG. 15, the grip 1320 may include at least one finger pocket 1322 disposed at least at the distal end region 1332 and/or the proximal end region 1334 of the impermeable fluid barrier 1306. The finger pocket 1322 is configured for being fitted over a finger or digit of the hand of the user. The finger pocket 1322 may be configured to be worn on a single digit or two digits of a hand. In some embodiments, the finger pocket 1322 may include a material that is needle resistant. As such, the covering of at least a portion of the finger with the needle-resistant material may further reduce needle stick injuries. In some embodiments, the finger pocket 1322 can include any suitable fluid imporous material(s), such as a fluid impermeable polymer (e.g., silicone, polypropylene, polyethylene, polyethylene terephthalate, a polycarbonate, etc.), a metal film, natural rubber, another suitable material, or combinations thereof. In some embodiments, the finger pocket 1322 may be sized to be used on more than one digit. In other words, the user may desire to use a certain digit for a tool or other reason. The user may slide a digit that was previously within the finger pocket 1322 out of the pocket and another digit into the pocket. With a finger pocket 1322, the user has the ability to alternate the finger, the angle of the fluid collection assembly 1300 on the finger, use the fluid collection assembly 1300 temporarily without a finger in the finger pocket 1322, etc. Additionally, as needed, the user can exchange control of the fluid collection assembly 1300 with another user, who could then place a finger within the finger pocket 1322 without readjustment or difficulty. The finger pocket 1322 can be closed end as shown in FIGS. 13A-13B or open ended as shown in FIG. 14.

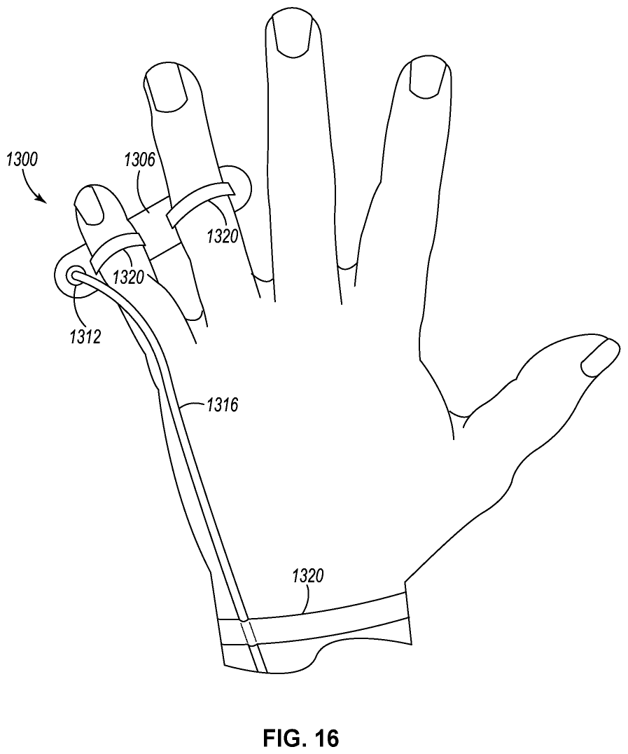

In some embodiments, the grip 1320 may need to be secured to the finger securely. As shown in FIG. 16, the grip 1320 may include an elastic loop, a tie, a hook-and-loop fastener, or other suitable clasp. The grip 1320 may be adjustable or a predetermined size configured for the user's finger size, the user's preferences, and/or the surgery procedure. In some embodiments, the grip 1320 may further include other loops or fasteners to secure the conduit 1316 out of the way of the fingers and/or the surgical field. In some embodiments, the grip 1320 may include a wrist strap.

In some embodiments, a weight 1324 may be integrated into the fluid impermeable barrier 1306. The weight 1324 may counter-balance the conduit 1316 to make it easier for the user to control the fluid collection assembly 1300 and position the opening 1310 to draw the fluid into the chamber 1308. The weight 1324 can be any suitable weight and material and can be embedded into the fluid impermeable barrier 1306 during manufacturing. In some embodiments, the weight 1324 may be adjustable. The fluid collection assembly 1300 may include weights at various locations coupled to and/or integrated into the fluid impermeable barrier 1306. In some embodiments, the weight 1324 may be located at the distal end region 1332 of the fluid collection assembly 1300.

In some embodiments, the fluid collection assembly 1300 may include a vent 1326. The vent 1326 may relieve pressure accumulation from within the fluid impermeable barrier 1306 and can reduce clogging of the fluid collection assembly, discussed in more detail below. In some embodiments, the vent 1326 can be located near the grip 1320 and or in a location that does not prevent or reduce the suction of the blood through the opening 1310. The vent can be sized appropriately to allow the passage of gasses without allowing the passage of fluids. The vent can be blocked with a finger and/or a port if/when required. In some embodiments, the vent can include a valve. The vent can be configured to minimize both over pressurization and under pressurization as required for by the application of the fluid collection assembly 1300.

The fluid collection assembly 1300 may become clogged with blood as it is used. In some embodiments, the fluid impermeable barrier 1306 may include an anti-clogging protein disposed thereon to minimize the number of times the fluid collection assembly 1300 would need to be replaced during a surgery. In some embodiments the fluid impermeable barrier 1306 may be coated with heparin or any other suitable anti-clotting protein.

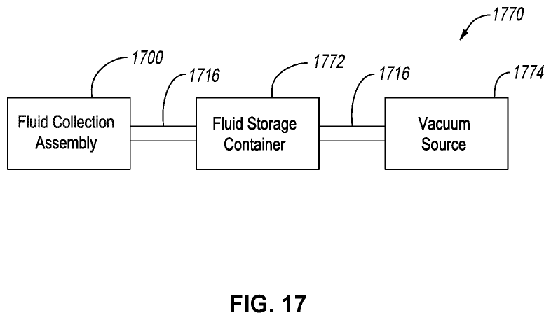

FIG. 17 is a block diagram of a fluid collection system 1770 for fluid collection, according to an embodiment. The fluid collection system 1770 includes a fluid collection assembly 1700, a fluid storage container 1772, and a vacuum source 1774. The fluid collection assembly 1700 may be the same or substantially similar to any of the fluid collection assemblies disclosed herein. The fluid collection assembly 1700, the fluid storage container 1772, and the vacuum source 1774 may be fluidly coupled to each other via one or more conduits 1716. For example, fluid collection assembly 1700 may be operably coupled to one or more of the fluid storage container 1772 or the vacuum source 1774 via the conduit 1716. The bodily fluids collected in the fluid collection assembly 1700 may be removed from the fluid collection assembly 1700 via the conduit 1716 which protrudes into the fluid collection assembly 1700. For example, an inlet of the conduit 1716 may extend into the fluid collection assembly 1700, such as to a reservoir therein. The outlet of the conduit 1716 may extend into the fluid collection assembly 1700 or the vacuum source 1774. Suction force may be introduced into the chamber of the fluid collection assembly 1700 via the inlet of the conduit 1716 responsive to suction (e.g., vacuum) force applied at the outlet of the conduit 1716.

The suction force may be applied to the outlet of the conduit 1716 by the vacuum source 1774 either directly or indirectly. The suction force may be applied indirectly via the fluid storage container 1772. For example, the outlet of the conduit 1716 may be disposed within the fluid storage container 1772 and an additional conduit 1716 may extend from the fluid storage container 1772 to the vacuum source 1774. Accordingly, the vacuum source 1774 may apply suction to the fluid collection assembly 1700 via the fluid storage container 1772. The suction force may be applied directly via the vacuum source 1774. For example, the outlet of the conduit 1716 may be disposed within the vacuum source 1774. An additional conduit 1716 may extend from the vacuum source 1774 to a point outside of the fluid collection assembly 1700, such as to the fluid storage container 1772. In such examples, the vacuum source 1774 may be disposed between the fluid collection assembly 1700 and the fluid storage container 1772.

The fluid storage container 1772 is sized and shaped to retain bodily fluids therein. The fluid storage container 1772 may include a bag (e.g., drainage bag), a bottle or cup (e.g., collection jar), or any other enclosed container for storing bodily fluids such as urine. In some examples, the conduit 1716 may extend from the fluid collection assembly 1700 and attach to the fluid storage container 1772 at a first point therein. An additional conduit 1716 may attach to the fluid storage container 1772 at a second point thereon and may extend and attach to the vacuum source 1774. Accordingly, a vacuum (e.g., suction) may be drawn through fluid collection assembly 1700 via the fluid storage container 1772. Bodily fluids, such as urine, may be drained from the fluid collection assembly 1700 using the vacuum source 1774.

The vacuum source 1774 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 1774 may provide a vacuum or suction to remove bodily fluids from the fluid collection assembly 1700. In some examples, the vacuum source 1774 may be powered by one or more of a power cord (e.g., connected to a power socket), one or more batteries, or even manual power (e.g., a hand operated vacuum pump). In some examples, the vacuum source 1774 may be sized and shaped to fit outside of, on, or within the fluid collection assembly 1700. For example, the vacuum source 1774 may include one or more miniaturized pumps or one or more micro pumps. The vacuum sources 1774 disclosed herein may include one or more of a switch, a button, a plug, a remote, or any other device suitable to activate the vacuum source 1774.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. A fluid collection assembly, comprising:
a fluid impermeable barrier defining at least a chamber, at least one opening, a fluid outlet, and a sump, the fluid impermeable barrier including a distal end region and a proximal end region;
a fluid permeable support;
a fluid permeable membrane disposed on at least a portion of the fluid permeable support, the fluid permeable membrane extending across the at least one opening; and
a conduit connected to the fluid outlet, the conduit including an inlet disposed in or adjacent to the sump;
wherein the fluid collection assembly exhibits a relatively thin shape.

2. The fluid collection assembly of claim 1, wherein the relatively thin shape is of a generally rectangular prism shape.

3. The fluid collection assembly of claim 1, wherein the relatively thin shape includes a plurality of branches extending from at least one common area, and wherein the plurality of branches form a gap therebetween.

4. The fluid collection assembly of claim 3, wherein the plurality of branches and the at least one common area form a generally in-plane bend.

5. The fluid collection assembly of any one of claim 3 or 4, wherein the plurality of branches and the at least one common area form a generally V-like shape.

6. The fluid collection assembly of any one of claim 3 or 4, wherein the plurality of branches and the at least one common area form a generally Y-like shape.

7. The fluid collection assembly of any one of claim 3 or 4, wherein the at least one common area includes a plurality of common areas and at least one of the plurality of branches extends between at least two of the plurality of common areas.

8. The fluid collection assembly of claim 7, wherein the relatively thin shape forms a generally zig-zag shape.

9. The fluid collection assembly of claim 8, wherein the plurality of branches extends from the plurality of common area in a direction that is generally parallel to each other.

10. The fluid collection assembly of claim 8, wherein the plurality of branches extends from the plurality of common area in a direction that is generally obliquely angled relative to each other.

11. The fluid collection assembly of claim 8, wherein the plurality of branches includes two branches and the plurality of common areas includes two common areas, and wherein each of the two branches extend between the two common areas.

12. The fluid collection assembly of claim 3, wherein the fluid impermeable barrier defines the gap.

13. The fluid collection assembly of any one of claims 1-12, wherein the fluid permeable support exhibits a generally rectangular cross-sectional shape.

14. The fluid collection assembly of any one of claims 1-12, wherein the fluid permeable support exhibits a generally trapezoidal cross-sectional shape.

15. The fluid collection assembly of any one of claims 1-14, wherein the opening is an elongated opening exhibiting a length and a width measured perpendicular to the length, the length is greater than the width, and wherein the length is about 3 cm to about 30 cm and the width is about 1 cm to about 5 cm.

16. The fluid collection assembly of any one of claims 1-15, wherein the fluid outlet is spaced from the distal end region and the proximal end region of the fluid impermeable barrier.

17. The fluid collection assembly of any one of claims 1-16, wherein the sump is spaced from the distal end region and the proximal end region of the fluid impermeable barrier.

18. The fluid collection assembly of any one of claims 1-17, wherein at least one of the fluid permeable support or the fluid permeable membrane define a bore generally parallel to a longitudinal axis of the fluid collection assembly, the conduit extending in the bore from the sump to the fluid outlet.

19. The fluid collection assembly of any one of claims 1-17, wherein at least one of the fluid permeable support or the fluid permeable membrane define a bore that is not generally parallel to a longitudinal axis of the fluid collection assembly, the conduit extending in the bore from the sump to the fluid outlet.

20. The fluid collection assembly of any one of claims 1-17, wherein the conduit extends from the fluid outlet along a portion of a back surface of the fluid impermeable barrier or the conduit extends within a portion of the fluid impermeable barrier that forms the back surface, the back surface of the fluid impermeable barrier is opposite the opening.

21. The fluid collection assembly of any one of claims 1-20, further comprising at least one grip coupled to the fluid impermeable barrier.

22. The fluid collection assembly of claim 21, wherein the at least one grip is coupled to a surface of the fluid impermeable barrier opposite the at least one opening.

23. The fluid collection assembly of any one of claim 21 or 22, wherein the at least one grip includes at least one finger pocket disposed at least at the distal end region or the proximal end region of the impermeable fluid barrier.

24. The fluid collection assembly of any one of claims 21-23, wherein the grip includes an elastic loop, a tie, or a hook-and-loop fastener.

25. The fluid collection assembly of any one of claims 1-24, wherein the fluid impermeable barrier includes an anti-clotting protein disposed thereon.

26. The fluid collection assembly of any one of claims 1-25, further comprising a weight integrated into the fluid impermeable barrier.

27. A fluid collection system, comprising:
the fluid collection assembly of any one of claims 1-26;
a fluid storage container; and
a vacuum source;
wherein the chamber of the fluid collection assembly is in fluid communication with the fluid storage container and the vacuum source via one or more conduits.
